(19) United States
(12) Patent Application Publication
(10) Pub. No.: US 2008/0200500 A1
(43) Pub. Date: Aug. 21, 2008

(54) Quinoline Derivatives as Nk3 Antagonists

(75) Inventors: Thomas R. Simpson, Wilmington, DE (US); James Kang, Wilmington, DE (US); S. Jeffrey Albert, Wilmington, DE (US); Cristobal Alhambra, Wilmington, DE (US); M. Gerard Koether, Wilmington, DE (US); M. James Woods, Wilmington, DE (US); Yan Li, Wilmington, DE (US)

Correspondence Address: ASTRA ZENECA PHARMACEUTICALS LP;GLOBAL INTELLECTUAL PROPERTY, 1800 CONCORD PIKE, WILMINGTON, DE, 19850-5437, US

(73) Assignee: AstraZeneca AB, Sodertalje (SE)

(21) Appl. No.: 11/914,583

(22) PCT Filed: May 30, 2006
(86) PCT No.: PCT/SE2006/000630

§ 371 Date: Mar. 11, 2008

Related U.S. Application Data
(60) Provisional application No. 60/687,418, filed on Jun. 3, 2005.

(51) Int. Cl. A61K 31/472; C07D 215/52; A61P 25/18; A61P 3/04; A61P 25/24; A61P 25/22

(52) U.S. Cl. 514/307; 546/169

(57) ABSTRACT

Compounds of Formula I

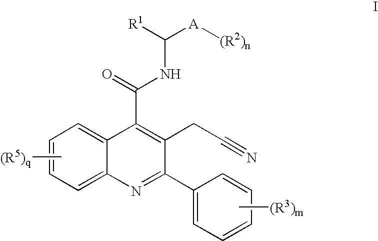

wherein R¹, A, R², n, R³, m, R⁵ and q are as described in the specification, pharmaceutically-acceptable salts, methods of making, pharmaceutical compositions containing and methods for using the same.

### FIELD OF THE INVENTION

Described herein are quinoline derivatives, pharmaceutical compositions comprising them, and the use of such compounds in the treatment of peripheral and central nervous system diseases or disorders.

### BACKGROUND OF THE INVENTION

Anxiety, depression, schizophrenia and obesity daily affect many millions of people. These conditions are thought to be brain disorders that have serious and lasting effects on people's lives and impact the sufferers and their friends and relatives.

People with schizophrenia often have trouble thinking clearly or making decisions. They may have a hard time telling real life from fantasy. They may have so-called positive symptoms such as delusions or hallucinations which they experience but which do not reflect reality, and see or believe things that are not real; or they may have negative symptoms and lack behaviors or feelings that normal people have, avoid social contact and be emotionally withdrawn. Often they start to do things, but not follow through and take no pleasure or interest in life; they may be confused in thinking and speech and act in ways that do not make sense.

People who have generalized anxiety disorder (GAD) worry excessively and uncontrollably about everyday things. This constant worry affects daily functioning and can cause physical symptoms can include sweating, nausea, gastrointestinal discomfort or diarrhea. Sufferers tend to be irritable and complain about feeling on edge, are easily tired and have trouble sleeping. GAD can occur with other anxiety disorders, depressive disorders, or substance abuse. The intensity, duration and frequency of worrying varies but is disproportionate to the issue and interferes with the sufferer's performance of tasks and ability to concentrate.

Depressive disorder is an illness that involves the body, mood, and thoughts. It affects the way a person eats and sleeps, the way they feel about themselves, and the way they think about things. People with a depressive illness cannot merely “pull themselves together” and get better. Without treatment, symptoms can last for weeks, months, and even years. Major depression interferes with a persons ability to work, study, sleep, eat, and enjoy life. A disabling episode of depression may occur only once but more commonly occurs several times in a lifetime. A less severe type of depression, termed dysthymia, involves long-term, chronic symptoms that do not disable, but keep one from functioning well or from feeling good. Many people with dysthymia also experience major depressive episodes at some time in their lives. Bipolar disorder is yet another type of depression that is also called manic-depressive illness. It is not as prevalent as other forms of depressive disorders and bipolar disorder is characterized by cycling mood changes that swing between manic highs and depressive lows. Sometimes the mood switches are dramatic and rapid, but most often they are gradual. When in the depressed phase, a person can have any or all of the symptoms of a depressive disorder. When in the manic phase, a person may be overactive, overtalkative, and have a great deal of energy. Manic persons often think differently and their judgment and social behavior changes in ways that cause serious problems and embarrassment; they may feel elated, have grand schemes, make unwise business decisions and indulge in romantic sprees. Untreated mania can also evolve into a psychotic state.

Obese persons work in most occupations and businesses and being obese may cause little or no inconvenience in a person's life. Over time, however, obesity may cause discomfort, or even bodily pain, and affect normal daily activities. A person with severe obesity may find their ability to perform their chosen occupation so compromised that they qualify for disability. Additionally, obesity is an intractable condition for which many seek treatment.

Collectively, anxiety, depression, schizophrenia and obesity daily affect many millions of people and effective treatment for these conditions is a largely unmet need.

Tachykinin receptors are the targets of a family of structurally related peptides which include substance P (SP), neurokinin A (NKA) and neurokinin B (NKB), collectively “tachykinins.” Tachykinins are synthesized in the central nervous system (CNS), and peripheral tissues, where they exert a variety of biological activities. Three tachykinin receptors are known which are named neurokinin-1 (NK-1), neurokinin-2 (NK-2) and neurokinin-3 (NK-3) receptors. NK-1 and NK-2 receptors are expressed in a wide variety of peripheral tissues and NK-1 receptors are also expressed in the CNS whereas NK-3 receptors are primarily expressed in the CNS.

The neurokinin receptors mediate a variety of tachykinin-stimulated biological effects that include: transmission of excitatory neuronal signals in the CNS and periphery (e.g. pain signals), modulation of smooth muscle contractile activity, modulation of immune and inflammatory responses, induction of hypotensive effects via dilation of the peripheral vasculature, and stimulation of endocrine and exocrine gland secretions.

In the CNS, activation of NK-3 receptors has been shown to modulate dopamine, acetylcholine and serotonin release, suggesting a therapeutic utility for NK-3 ligands for the treatment of a variety of disorders including anxiety, depression, schizophrenia and obesity. Studies in primate brain have shown the presence of NK-3 mRNA in a variety of regions relevant to these disorders. Studies in rats have shown NK-3 receptors to be located on MCH-containing neurons in the lateral hypothalamus and zona incerta, again suggesting a therapeutic utility for NK-3 ligands for obesity.

Non-peptide ligands have been developed for each of the tachykinin receptors, however known non-peptide NK-3 receptor antagonists suffer from a number of problems such as species selectivity which limits the potential to evaluate these compounds in many appropriate disease models. New non-peptide NK-3 receptor ligands are therefore desirable for use as therapeutic agents and as tools to investigate the biological consequences of NK-3 receptor modulation.

### SUMMARY OF THE INVENTION

Disclosed are compounds, particularly quinoline derivatives with affinity for NK-3 receptors (NK-3r). These compounds have potential for the treatment of a broad array of diseases, disorders and conditions including but not limited to depression, anxiety, schizophrenia, cognitive disorders, psychoses, obesity, inflammatory diseases including irritable bowel syndrome and inflammatory bowel disorder, emesis, pre-eclampsia, chronic obstructive pulmonary disease, disorders associated with excessive gonadotrophins and/or androgens including dysmenorrhea, benign prostatic hyperplasia, prostatic cancer, and testicular cancer in which modulation of the activity of NK-3 receptors is beneficial.

Ligands for NK-3 receptors disclosed herein and stereoisomers, enantiomers, in vivo-hydrolysable precursors and pharmaceutically-acceptable salts thereof are compounds of Formula I,

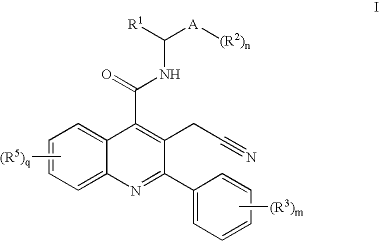

wherein:

R¹ is selected from H, C₁₋₆alkyl-, C₃₋₆cycloalkyl-, C₁₋₆alkyl-C(O)— and C₁₋₄alkylOC(O)—;

A is phenyl or C₃₋₇cycloalkyl-;

n is 1, 2 or 3;

R² at each occurrence is independently selected from H, —OH, —NH₂, —CN, halogen, C₁₋₆alkyl-, C₃₋₇cycloalkyl-, C₁₋₆alkoxy- and C₁₋₆alkoxyC₁₋₆alkyl-;

R³ at each occurrence is independently selected from H, —OH, —NH₂, —NO₂, —CN, halogen, C₁₋₆alkyl-, C₁₋₆alkoxy- and C₁₋₆alkoxyC₁₋₆alkyl-;

m is 1, 2 or 3;

R⁵ at each occurrence is independently selected from H, —OH, —CN, halogen, —R⁶, —OR⁶, —NR⁶R⁷, —SR⁶, —SOR⁶ and —SO₂R⁶;

q is 1, 2 or 3;

wherein:

R⁶ and R⁷ at each occurrence are independently selected from H, a C₁₋₆ straight or branched alkyl group, a C₂₋₆ straight or branched alkenyl or alkynyl group and a C₃₋₇carbocyclic group having zero, one or two double- or triple-bonds, wherein said groups are either unsubstituted or substituted with one or more moieties selected from —OH, ═O, —NH₂, —CN, halogen, aryl and C₁₋₃alkoxy-;

and,

when R¹, R² or R³ is an alkyl, cycloalkyl, alkoxy or alkoxyalkyl moiety, said moieties are unsubstituted or have 1, 2, 3, 4 or 5 substituents independently selected at each occurrence from —OH, —NH₂, —CN, phenyl and halogen.

Also disclosed are pharmaceutical compositions and formulations containing the compounds, methods of using them to treat diseases and conditions either alone or in combination with other therapeutically-active compounds or substances, processes and intermediates used to prepare them, uses of them as medicaments, uses of them in the manufacture of medicaments and uses of them for diagnostic and analytic purposes. In particular are disclosed compounds, compositions containing them, and methods using them for treating or preventing conditions and disorders associated with a wide range of diseases or disorders in which NK-3 receptors are considered to have a role.

### DETAILED DESCRIPTION OF THE INVENTION

Compounds described herein are compounds of Formula I

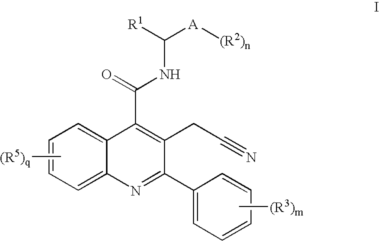

wherein:

R¹ is selected from H, C₁₋₆alkyl-, C₃₋₆cycloalkyl-, C₁₋₆alkyl-C(O)— and C₁₋₄alkylOC(O)—;

A is phenyl or C₃₋₇cycloalkyl-;

n is 1, 2 or 3;

R² at each occurrence is independently selected from H, —OH, —NH₂, —CN, halogen, C₁₋₆alkyl-, C₃₋₇cycloalkyl-, C₁₋₆alkoxy- and C₁₋₆alkoxyC₁₋₆alkyl-;

R³ at each occurrence is independently selected from H, —OH, —NH₂, —NO₂, —CN, halogen, C₁₋₆alkyl-, C₁₋₆alkoxy- and C₁₋₆alkoxyC₁₋₆alkyl-;

m is 1, 2 or 3;

R⁵ at each occurrence is independently selected from H, —OH, —CN, halogen, —R⁶, —OR⁶, —NR⁶R⁷, —SR⁶, —SOR⁶ and —SO₂R⁶;

q is 1, 2 or 3;

wherein:

R⁶ and R⁷ at each occurrence are independently selected from H, a C₁₋₆ straight or branched alkyl group, a C₂₋₆ straight or branched alkenyl or alkynyl group and a C₃₋₇carbocyclic group having zero, one or two double- or triple-bonds, wherein said groups are either unsubstituted or substituted with one or more moieties selected from —OH, ═O, —NH₂, —CN, halogen, aryl and C₁₋₃alkoxy-;

and,
when R¹, R² or R³ is an alkyl, cycloalkyl, alkoxy or alkoxyalkyl moiety, said moieties are unsubstituted or have 1, 2, 3, 4 or 5 substituents independently selected at each occurrence from —OH, —NH₂, —CN, phenyl and halogen,

stereoisomers, enantiomers, in vivo-hydrolysable precursors and pharmaceutically-acceptable salts thereof.

Some compounds are those in accord with Formula I, wherein:

A is phenyl;

R¹ is selected from C₁₋₆alkyl-, C₃₋₆cycloalkyl-, or C₁₋₆allyl-O—C(O)—;

n at each occurrence is independently selected from 1 or 2;

stereoisomers, enantiomers, in vivo-hydrolysable precursors and pharmaceutically-acceptable salts thereof.

Other compounds are those in accord with Formula I, wherein:

A is phenyl;

R¹ is selected from C₁₋₆alkyl or —(CO)—O—C₁₋₆alkyl;

n at each occurrence is 1;

stereoisomers, enantiomers, in vivo-hydrolysable precursors and pharmaceutically-acceptable salts thereof.

Yet other compounds are those in accord with Formula II

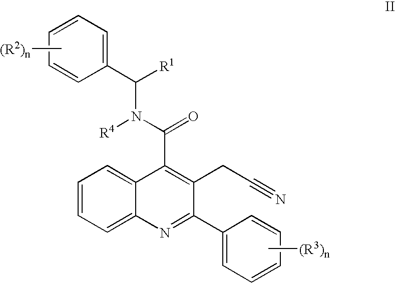

wherein R⁴ is H and R¹, R² and R³ are as defined for Formula I and n and m are selected from 1, 2, 3, 4 or 5, and

stereoisomers, enantiomers, in vivo-hydrolysable precursors and pharmaceutically-acceptable salts thereof.

Still other compounds are those in accord with Formula III

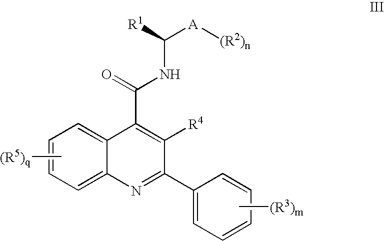

wherein R¹, A, R², n, R³, m, R⁴, R⁵ and q are as defined for Formula I.

Particular compounds of Formula I are:
3-(cyanomethyl)-2-phenyl-N-[(1S)-1-phenylpropyl]quinoline-4-carboxamide;
3-(cyanomethyl)-2-phenyl-N-[(1S)-1-phenylethyl]quinoline-4-carboxamide; methyl(2R)-({[3-(cyanomethyl)-2-phenylquinoline-4-yl]carbonyl}amino)(phenyl)acetate;
3-(cyanomethyl)-N-[(S)-cyclopropyl(3-fluorophenyl)methyl]-2-phenylquinoline-4-carboxamide;
3-(cyanomethyl)-2-(3-fluorophenyl)-N-[(1S)-1-phenylpropyl]quinoline-4-carboxamide, and
3-(cyanomethyl)-N-[(S)-cyclopropyl(3-fluorophenyl)methyl]-2-(3-fluorophenyl)quinoline-4-carboxamide;

stereoisomers, enantiomers, in vivo-hydrolysable precursors and pharmaceutically-acceptable salts thereof.

Disclosed compounds have the advantage that they may be more soluble, be more easily absorbed and more efficacious in vivo, produce fewer side effects, be less toxic, be more potent, more selective, be longer acting, be less metabolized and/or have a better pharmacokinetic profile than, or have other useful pharmacological or physicochemical properties over known compounds. Using assays for functional activity described herein, compounds described herein will be found to have IC50's of less than about 1 μM for NK-3 receptors and many compounds will be found to have IC50's of less than about 100 nM for NK-3 receptors. The disclosure of U.S. Provisional Application 60/687,418 is incorporated herein in its entirety.

### Abbreviations and Definitions

As used herein, unless otherwise indicated, C₁₋₆alkyl includes but is not limited to methyl, ethyl, n-propyl, n-butyl, i-propyl, i-butyl, t-butyl, s-butyl moieties, whether alone or part of another group and alkyl groups may be straight-chained or branched.

As used herein, unless otherwise indicated, C₁₋₆alkoxy includes but is not limited to —O-methyl, —O-ethyl, —O-n-propyl, —O-n-butyl, —O-i-propyl, —O-i-butyl, —O-t-butyl, —O-s-butyl moieties, whether alone or part of another group and alkoxy groups may be straight-chained or branched.

As used herein C₃₋₆cycloalkyl groups include but are not limited to the cyclic alkyl moieties cyclopropyl, cyclobutyl, cyclopentyl and cyclohexyl.

As used herein, unless otherwise indicated, C₂₋₆alkenyl includes but is not limited to 1-propenyl, 2-propenyl, 1-butenyl, 2-butenyl and 3-butenyl.

As used herein, unless otherwise indicated, C₂₋₆alkynyl includes but is not limited to ethynyl, 1-propynyl, 2-propynyl, 1-butynyl, 2-butynyl and 3-butynyl.

As used herein, unless otherwise indicated, halo or halogen refers to fluorine, chlorine, bromine, or iodine;

As used herein, aryl includes to phenyl and naphthyl;

As used herein, aromatic or non-aromatic heterocyclic rings include but are not limited to N— or C-linked furyl, imidazolyl, oxazolyl, pyrrolidinyl, thiazolyl, thiophenyl, pyrrolyl, morpholinyl, piperidinyl, piperazinyl, pyrazinyl, pyridyl, pyrimidinyl, indanyl, indolyl, quinolinyl, isoquinolinyl, quinazolinyl, quinoxalinyl, benzo[b]thiophenyl, benzoxazolyl, or benzthiazolyl;

DMF referes to dimethylformamide

THF refers to tetrahydrofuran

HOBT refers to 1-hydroxybenzotriazole

DCM refers to dichloromethane;

EtOAc refers to ethyl acetate;

EDC refers to 1-(3-dimethylaminopropyl)-3-ethylcarbodiimide;

EDTA refers to ethylenediaminetetraacetic acid;

HEPES refers to 4-(2-hydroxyethyl)-1-piperazine ethane sulfonic acid, monosodium salt, and

TEA refers to triethylamine.

In processes described herein, RT means room temperature, h means hours and other abbreviations have their conventional meanings.

In processes described herein, where necessary, hydroxy, amino, or other reactive groups may be protected using a protecting group as described in the standard text “Protecting groups in Organic Synthesis”, 3^{rd} Edition (1999) by Greene and Wuts.

Unless otherwise stated, reactions are conducted under an inert atmosphere, preferably under a nitrogen atmosphere and are usually conducted at a pressure of about one to about three atmospheres, preferably at ambient pressure (about one atmosphere).

The compounds and intermediates may be isolated from their reaction mixtures by standard techniques.

Acid addition salts of the compounds of Formula I which may be mentioned include salts of mineral acids, for example the hydrochloride and hydrobromide salts; and salts formed with organic acids such as formate, acetate, maleate, benzoate, tartrate, and fumarate salts.

Acid addition salts of compounds of Formula I may be formed by reacting the free base or a salt, enantiomer or protected derivative thereof, with one or more equivalents of the appropriate acid. The reaction may be carried out in a solvent or medium in which the salt is insoluble or in a solvent in which the salt is soluble, e.g., water, dioxane, ethanol, tetrahydrofuran or diethyl ether, or a mixture of solvents, which may be removed in vacuum or by freeze drying. The reaction may be a metathetical process or it may be carried out on an ion exchange resin.

Certain compounds of Formula I may exist in tautomeric or enantiomeric forms, all of which are within the scope of Formula I. The various optical isomers may be isolated by separation of a racemic mixture of the compounds using conventional techniques, e.g. fractional crystallization, or chiral HPLC. Alternatively the individual enantiomers may be made by reaction of the appropriate optically active starting materials under reaction conditions which will not cause racemization.

### Synthesis and Schemes

Compounds of Formula I wherein A is phenyl may be made by the method illustrated in Scheme A.

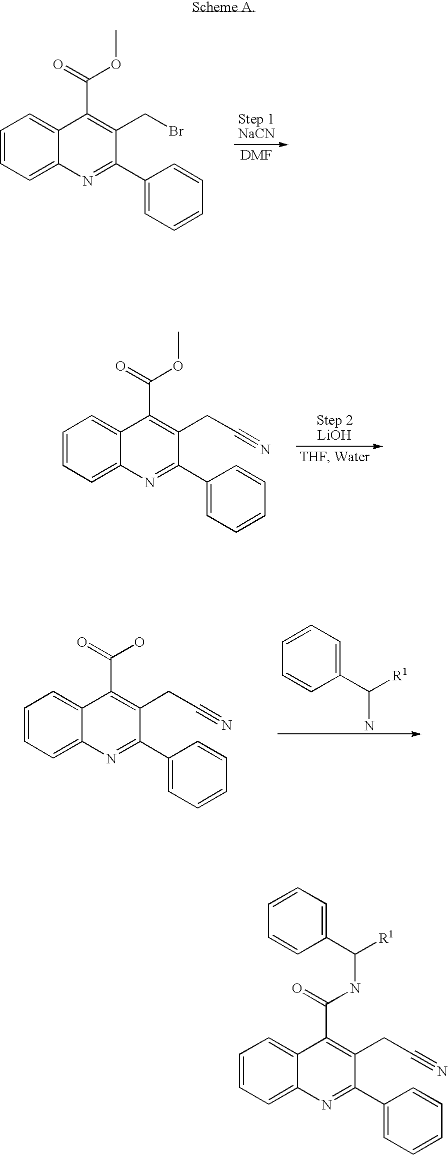

Synthesis of the nitrile ester product of Step 1 can be accomplished by reacting the appropriate bromomethyl substituted quinoline with sodium cyanide in DMF. This nitrile ester can then be converted to the acid as shown in Step 2 by reacting with LiOH in THF/water solvent. The acid can then be coupled to the appropriate amine by reacting with EDC (N-(3-dimethylaminopropyl)-N′-ethylcarbodiimide), HOBt (hydroxybenzotriazole) and morpholine in a CH₂Cl₂ solution.

Compounds wherein A is C₃₋₇cycloalkyl may be made by processes analogous to those illustrated in Scheme 1 as may compounds wherein A is substituted phenyl or C₃₋₇cycloalkyl

Compounds of Formulae I, II or III may be made by a process comprising: coupling an acid

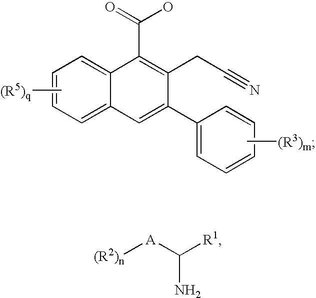

to an amine by reaction with EDC (N-(3-dimethylaminopropyl)-N′-ethylcarbodiimide), HOBt (hydroxybenzotriazole) and morpholine in a CH₂Cl₂ solution to form said compound of Formula I wherein R¹, A, R², n, R³, m, R⁵ and q are as described in the specification Other compounds of Formula I, II or III may be made by a process comprising: reacting a bromomethyl substituted quinoline ester

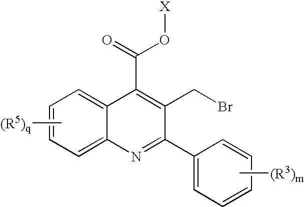

where X is an alkyl moiety, with sodium cyanide in DMF to form a nitrile

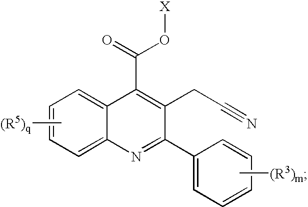

reacting said nitrile with LiOH in THF/water solvent to form an acid

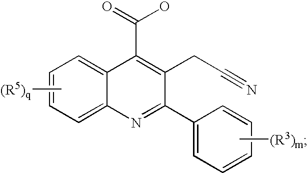

coupling said acid to an amine

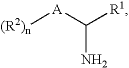

by reacting with EDC (N-(3-dimethylaminopropyl)-N′-ethylcarbodiimide), HOBt (hydroxybenzotriazole) and morpholine in a CH₂Cl₂ solution to form said compound of Formula I wherein R¹, A, R², n, R³, m, R⁵ and q are as described in the specification

An example compound, methyl 2-(3-(cyanomethyl)-2-phenylquinoline-4-carboxamido)-2-phenylacetate,

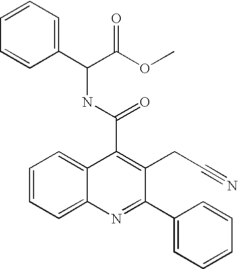

may be prepared by the method of Scheme A by reacting 3-bromomethyl-2-phenyl-quinoline-4-carboxylic acid methyl ester with sodium cyanide in DMF to form 3-cyanomethyl-2-phenyl-quinoline-4-carboxylic acid methyl ester. The 3-cyanomethyl-2-phenyl-quinoline-4-carboxylic acid methyl ester may then be converted to an acid by reacting with LiOH in a THF/water solvent. Formed 3-cyanomethyl-2-phenyl-quinoline-4-carboxylic acid may then be reacted with amino-phenyl-acetic acid methyl ester with EDC, HOBt and morpholine in a CH₂Cl₂ solution to form the title compound.

Compounds of Example 1 through 6 and other compounds of Formula I herein may be prepared as specifically described herein or by processes analogous to those described herein by use of suitable amines. Those of skill in the art will readily appreciate that many suitable amines may be used to form compounds within the scope of the subject matter described herein as Formula I

### Radiolabeled Compounds:

In a further aspect compounds described herein are those wherein one or more of the atoms is a radioisotope of the same element. In a particular form of this aspect the compound is labeled with tritium. Such radiolabeled compounds are synthesized either by incorporating radio-labeled starting materials or, in the case of tritium, exchange of hydrogen for tritium by known methods. Known methods include (1) electrophilic halogenation, followed by reduction of the halogen in the presence of a tritium source, for example, by hydrogenation with tritium gas in the presence of a palladium catalyst, or (2) exchange of hydrogen for tritium performed in the presence of tritium gas and a suitable organometallic (e.g. palladium) catalyst.

Compounds labeled with tritium are useful for the discovery of novel medicinal compounds which bind to and modulate the activity, by agonism, partial agonism, or antagonism, of an NK-3 receptor. Such tritium-labeled compounds may be used in assays that measure the displacement of such compounds to assess the binding of ligands that bind to NK-3 receptors.

In a further aspect compounds described herein additionally comprise one or more atoms of a radioisotope. In a particular form of this aspect the compound comprises a radioactive halogen. Such radio-labeled compounds are synthesized by incorporating radio-labeled starting materials by known methods. Particular embodiments of this aspect are those in which the radioisotope is selected from ¹⁸F, ¹²³I, ¹²⁵I, ¹³¹I, ⁷⁵Br, ⁷⁶Br, ⁷⁷Br or ⁸²Br. A most particular embodiment of this aspect is that in which the radioisotope is ¹⁸F. Such compounds comprising one or more atoms of a radioisotope are useful as positron emission tomography (PET) ligands and for other uses and techniques to determine the location of NK3 receptors.

### Therapeutic Uses of Compounds

Another aspect relates to the use of compounds in accord with Formula I in therapy and in compositions useful for therapy.

Another aspect encompasses the use of compounds described herein for the therapy of diseases mediated through the action of NK-3 receptors. Such an aspect encompasses methods of treatment or prophylaxis of diseases or conditions in which modulation of the NK-3 receptor is beneficial which methods comprise administering a therapeutically-effective amount of an antagonistic compound described herein to a subject suffering from said disease or condition.

One embodiment of this aspect is a method of treatment or prophylaxis of disorders, wherein the disorder is depression, anxiety, schizophrenia, cognitive disorders, psychoses, obesity, inflammatory diseases including irritable bowel syndrome and inflammatory bowel disorder, emesis, pre-eclampsia, chronic obstructive pulmonary disease, disorders associated with excessive gonadotrophins and/or androgens including dysmenorrhea, benign prostatic hyperplasia, prostatic cancer, or testicular cancer comprising administering a pharmacologically effective amount of a compound of Formula I to a patient in need thereof.

A further aspect is the use of a compound according to Formula I, an enantiomer thereof or a pharmaceutically-acceptable salt thereof, for the treatment or prophylaxis of a disease or condition in which modulation of the NK-3 receptor is beneficial. Particular diseases and conditions that may be treated are depression, anxiety, schizophrenia, cognitive disorders, psychoses, obesity, inflammatory diseases including irritable bowel syndrome and inflammatory bowel disorder, emesis, pre-eclampsia, chronic obstructive pulmonary disease, disorders associated with excessive gonadotrophins and/or androgens including dysmenorrhea, benign prostatic hyperplasia, prostatic cancer, and testicular cancer. More particular embodiments encompass uses of a compound for treatment or prophylaxis of anxiety, depression, schizophrenia and obesity. A further aspect is the use of a compound according to Formula I, an enantiomer thereof or a pharmaceutically-acceptable salt thereof, in the manufacture of a medicament for the treatment or prophylaxis of the diseases or conditions mentioned herein.

A particular embodiment of this aspect is the use of a compound described herein in the manufacture of a medicament for treatment or prophylaxis of depression, anxiety, schizophrenia, cognitive disorders, psychoses, obesity, inflammatory diseases including irritable bowel syndrome and inflammatory bowel disorder, emesis, pre-eclampsia, chronic obstructive pulmonary disease, disorders associated with excessive gonadotrophins and/or androgens including dysmenorrhea, benign prostatic hyperplasia, prostatic cancer, and testicular cancer.

### Pharmaceutical Compositions

Compounds of Formula I, enantiomers thereof, and pharmaceutically-acceptable salts thereof, may be used on their own or in the form of appropriate medicinal preparations for enteral or parenteral administration. Thus, according to a further aspect, there is provided a pharmaceutical composition including preferably less than 80% and more preferably less than 50% by weight of a compound described herein in admixture with an inert pharmaceutically-acceptable diluent, lubricant or carrier.

### Examples of Diluents, Lubricants and Carriers are:

for tablets and dragees: lactose, starch, talc, stearic acid;
for capsules: tartaric acid or lactose;
for injectable solutions: water, alcohols, glycerin, vegetable oils;
for suppositories: natural or hardened oils or waxes.

Such a pharmaceutical composition may be prepared by a process that comprises mixing or compounding the ingredients together and forming the mixed ingredients into tablets or suppositories or other administrable form, encapsulating the ingredients in capsules or dissolving the ingredients to form injectable solutions.

Pharmaceutically-acceptable derivatives include solvates and salts. For example, compounds of Formula I herein may form acid addition salts with acids, such as conventional pharmaceutically-acceptable acids including maleic, hydrochloric, hydrobromic, phosphoric, acetic, fumaric, salicylic, citric, lactic, mandelic, tartaric and methanesulfonic acids. Acid addition salts of the compounds of Formula I which may be mentioned include salts of mineral acids, for example the hydrochloride and hydrobromide salts; and salts formed with organic acids such as formate, acetate, maleate, benzoate, tartrate, and fumarate salts. Acid addition salts of compounds of Formula I may be formed by reacting the free base or a salt, enantiomer or protected derivative thereof, with one or more equivalents of the appropriate acid. The reaction may be carried out in a solvent or medium in which the salt is insoluble or in a solvent in which the salt is soluble, e.g., water, dioxane, ethanol, tetrahydrofuran or diethyl ether, or a mixture of solvents, which may be removed in vacuum or by freeze drying. The reaction may be a metathetical process or it may be carried out on an ion exchange resin.

For the uses, methods, medicaments and compositions mentioned herein the amount of compound used and the dosage administered will, of course, vary with the compound employed, the mode of administration and the treatment desired. However, in general, satisfactory results are obtained when compounds of Formula I are administered at a daily dosage of about 0.1 mg to about 20 mg/kg of animal body weight. Such doses may be given in divided doses 1 to 4 times a day or in sustained release form. For man, the total daily dose is in the range of from 5 mg to 1,400 mg, more preferably from 10 mg to 100 mg, and unit dosage forms suitable for oral administration comprise from 2 mg to 1,400 mg of the compound admixed with a solid or liquid pharmaceutical carriers, lubricants and diluents.

Some compounds of Formula I may exist in tautomeric, enantiomeric, stereoisomeric or geometric isomeric forms, all of which are included within the scope of the description. Optical isomers may be isolated by separation of a racemic mixture of the compounds using conventional techniques, e.g. fractional crystallization, or chiral HPLC. Alternatively the individual enantiomers may be made by reaction of the appropriate optically active starting materials under reaction conditions which will not cause racemization.

Exemplary compounds may be prepared by processes analogous to that described in Scheme 1. Those of skill in the art will readily appreciate that many suitable amines and acid chlorides and carboxylic acids may be used to form compounds within the scope of the subject matter described herein as Formula I.

### EXAMPLE COMPOUNDS

The compounds and processes are provided by way of illustration and example for clarity of understanding. However to those skilled in the art, upon contemplation of the teaching of compounds, processes and methods described herein, modifications and changes will be apparent that may be made thereto without departing from the spirit or scope of the description.

### Example 1. 3-(Cyanomethyl)-2-phenyl-N-(1S)-1-phenylpropyl]quinoline-4-carboxamide

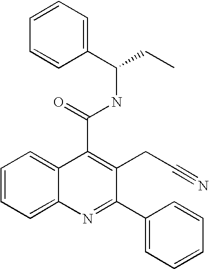

The title compound was prepared according to Scheme 1.

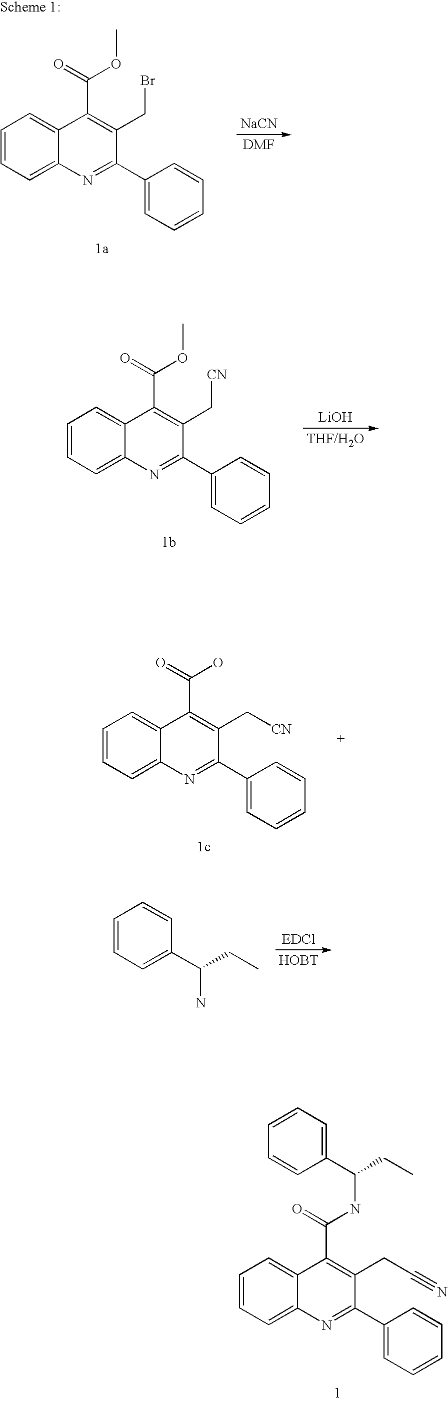

A solution of 3-(cyanomethy)-2-phenylquinoline-4-carboxylic acid (1c) (57.6 mg, 0.20 mmol), HOBT hydrate (46 mg, 0.30 mmol), 4-methylmorpholine (40 μL, 0.30 mmol) in methylene chloride (10 mL) was added EDC (58 mg, 0.30 mmol) at RT under N₂. (S)-1-phenyl propylamine (25.4 mg, 0.21 mmol) was then added and the reaction mixture stirred at RT for 12 h. The reaction mixture was further diluted with methylene chloride (30 mL) and washed successively with 5% citric acid, 10% aqueous sodium bicarbonate solution and brine. The organic phase was separated and dried over anhydrous sodium sulfate and then concentrated in vacuo. The residue was purified by chromatography eluting with 10-35% ethyl acetate/hexane to give the title compound (50 mg, 62%) as a light-yellow solid. ¹H NMR (300 MHz, CDCl₃) δ 0.96 (t, 3H), 2.01 (m, 2H), 4.67 (s, 2H), 5.29 (q, 1H), 6.50 (d, 1H), 7.32 (d, 2H), 7.34 (d, 2H), 7.39 (m, 1H), 7.78 (m, 2H), 7.84 (m, 2H), 8.08 (m, 1H), 8.30 (m, 2H), 8.42 (m, 2H). MS APCI, m/z=406 (M+1). LCMS: 2.30 min.

The starting acid, 3-(cyanomethy)-2-phenylquinoline-4-carboxylic acid (1c), was prepared in the following manner:

a) Methyl 3-(cyanomethyl)-2-phenylquinoline-4-carboxylate (1b)

To a solution of 3-(bromomethy)-2-phenylquinoline-4-carboxylate (1a) (356 mg, 1.0 mmol) in DMF (10 mL) was added sodium cyanide (54 mg, 1.1 mmol) and the reaction mixture stirred at RT for 12 h. All solvent was removed in vacuo and the residue was partitioned between ethyl acetate and 10% aqueous sodium bicarbonate solution, dried over sodium sulfate and then concentrated in vacuo. The residue was purified by chromatography eluting with 10-15% ethyl acetate/hexane to give the title compound (287 mg, 95%) as an off white solid. ¹H NMR (300 MHz, CDCl₃) δ 4.01 (s, 3H), 4.65 (s, 2H), 7.37 (m, 1H), 7.78 (m, 2H), 7.79 (m, 1H), 8.10 (m, 1H), 8.30 (m, 1H), 8.37 (m, 2H), 8.54 (m, 1H). MS APCI, m/z=303 (M+1). LCMS: 2.12 min.

b) 3-(Cyanomethyl)-2-phenylquinoline-4-carboxylic acid (1c)

To a solution of methyl 3-(cyanomethyl)-2-phenylquinoline-4-carboxylate (1b) (287 mg, 0.95 mmol) in THF (10 ml) was added the solution of lithium hydroxide monohydrate (46 mg, 1.9 mmol) in 5 ml water. The reaction mixture was stirred at RT for 12 h. The residue was acidified with 5% citric acid and extracted with ethyl acetate (50 ml×2). The organic phase was separated and washed with brine (20 ml) and dried over sodium sulfate and then concentrated in vacuo to afford the title compound (216 mg, 78.9%) as an off white solid. ¹H NMR (300 MHz, CDCl₃) δ 4.69 (s, 2H), 7.37 (m, 1H), 7.78 (m, 2H), 7.82 (m, 1H), 8.20 (m, 1H), 8.35 (m, 1H), 8.39 (m, 2H), 8.59 (m, 1H). MS APCI, m/z=289 (M+1). LCMS: 0.91 min.

### Example 2. 3-(Cyanomethyl)-2-phenyl-N-[(1S)-1-phenylethyl]quinoline-4-carboxamide

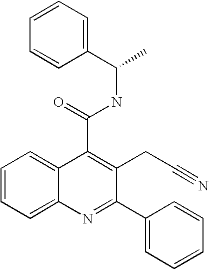

The title compound was prepared using a procedure similar to that described in Example 1, except using (1S)-1-phenylethanamine (25.4 mg, 0.21 mmol) as the amine component.

### Scheme 2:

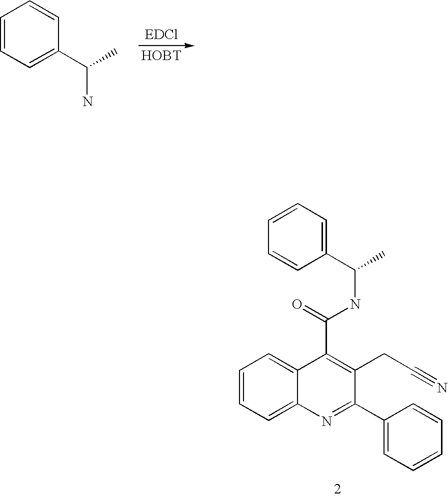

By the reaction shown in Scheme 2, the title compound (2) was obtained as a white solid (30 mg, 38%). ¹H NMR (300 MHz, CDCl₃) δ 1.56 (d, 3H), 4.73 (s, 2H), 5.39 (m, 1H), 6.48 (d, 1H), 7.31 (d, 2H), 7.34 (d, 2H), 7.39 (m, 1H), 7.78 (m, 2H), 7.84 (m, 2H), 8.08 (m, 1H), 8.30 (m, 2H), 8.42 (m, 2H). MS APCI, m/z=392 (M+1). LCMS: 2.21 min.

### Example 3. Methyl (2R)-({[3-(cyanomethyl)-2-phenylquinoline-4-yl]carbonyl}amino)(phenyl)acetate

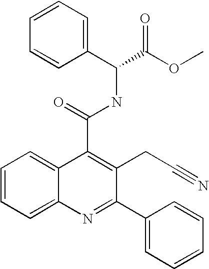

The title compound was prepared using a procedure similar to that described in Example 1, except using methyl (2R)-amino(phenyl)acetate (42.2 mg, 0.21 mmol) as the amine component.

### Scheme 3:

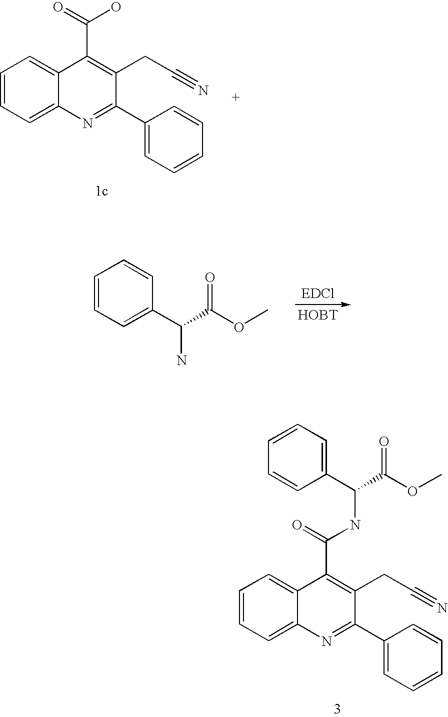

By the reaction shown in Scheme 3, the title compound (3) was obtained as a white solid (40 mg, 46%). ¹H NMR (300 MHz, CDCl₃) δ 3.78 (s, 3H), 3.83 (s, 2H), 5.87 (d, 1H), 6.72 (d, 1H), 6.74 (d, 2H), 7.04 (d, 2H), 7.06 (m, 1H), 7.37 (m, 2H), 7.41 (m, 2H), 7.76 (m, 1H), 7.84 (m, 2H), 8.04-8.19 (m, 2H). MS APCI, m/z=436 (M+1). LCMS: 2.20 min.

### Example 4. 3-(Cyanomethyl)-N-[(S)-cyclopropyl(3-fluorophenyl)methyl]-2-phenylquinoline-4-carboxamide

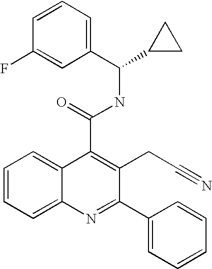

The title compound was prepared using a procedure similar to that described in Example 1, except using (S)-1-cyclopropyl-1-(3-fluorophenyl)methenamine hydrochloride (44.6 mg, 0.21 mmol) as the amine component.

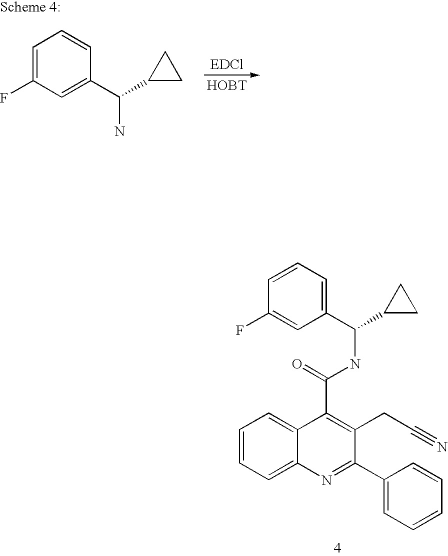

By the reaction shown in Scheme 4, the title compound (4) was obtained as a white solid (39.2 mg, 45%). ¹H NMR (300 MHz, CDCl₃) δ 0.52 (m, 2H), 0.70 (m, 2H), 1.60 (m, 1H), 4.67 (s, 2H), 5.03 (d, 1H), 6.86 (m, 1H), 6.94 (d, 1H), 7.16 (m, 1H), 7.24 (m, 1H), 7.30 (m, 1H), 7.39 (m, 1H), 7.76 (m, 2H), 7.84 (m, 2H), 8.04-8.19 (m, 4H). MS APCI, m/z=436 (M+1). LCMS: 2.37 min.

### Example 5. 3-(Cyanomethyl)-2-(3-fluorophenyl)-N-[(1S)-1-phenylpropyl]quinoline-4-carboxamide

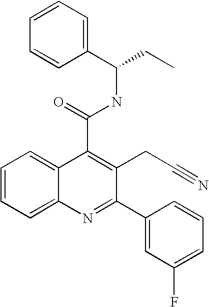

The title compound was prepared using a procedure similar to that described in Example 1, except using 3-(cyanomethyl)-2-(3-fluorophenyl)quinoline-4-carboxylic acid (61.2 mg, 0.2 mmol) as the acid component (1c′).

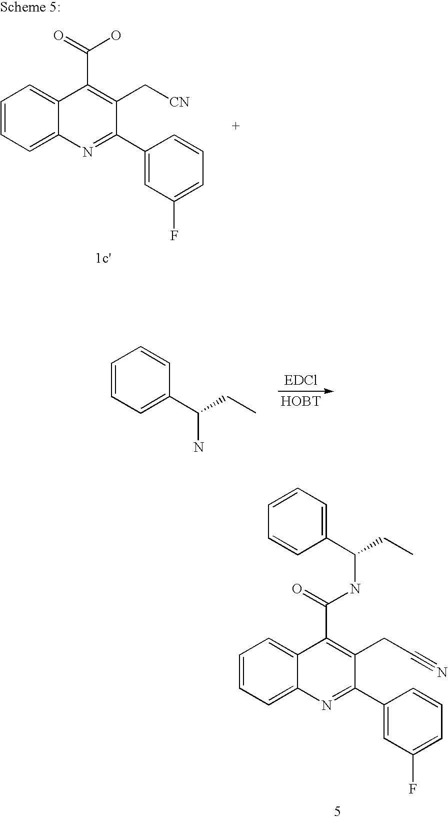

By the reaction shown in Scheme 5, the title compound (5) was obtained as a white solid (45 mg, 52.8%). ¹H NMR (300 MHz, CDCl₃) δ 0.96 (t, 3H), 2.0 (m, 2H), 4.62 (s, 2H), 5.25 (q, 1H), 5.72 (m, 1H), 6.81 (d, 1H), 7.20 (m, 1H), 7.32 (m, 1H), 7.35 (m, 1H), 7.80 (m, 1H), 7.85 (m, 1H), 8.08 (m, 2H), 8.14 (m, 1H), 8.30-8.36 (m, 4H). MS APCI, m/z=424 (M+1). LCMS: 2.37 min.

### Example 6. 3-(Cyanomethyl)-N-[(S)-cyclopropyl(3-fluorophenyl)methyl]-2-(3-fluorophenyl)quinoline-4-carboxamide

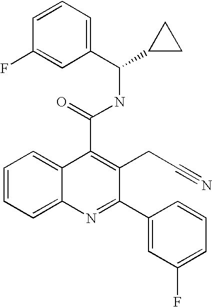

The title compound was prepared using a procedure similar to that described in Example 4, except using 3-(cyanomethyl)-2-(3-fluorophenyl)quinoline-4-carboxylic acid (61.2 mg, 0.2 mmol) as the acid component (1c′) and using (S)-1-cyclopropyl-1-(3-fluorophenyl)methenamine hydrochloride (44.6 mg, 0.21 mmol) as the amine component.

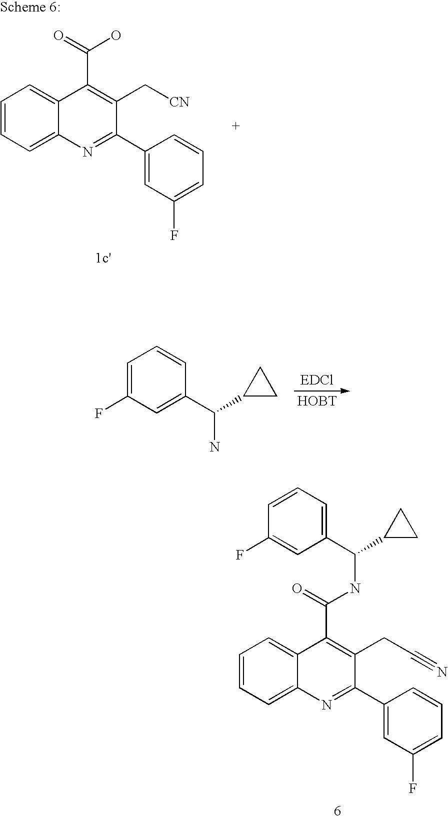

By the reaction shown in Scheme 6, the title compound (6) was obtained as a white solid (35 mg, 38.6%). ¹H NMR (300 MHz, CDCl₃) δ 0.52 (m, 2H), 0.70 (m, 2H), 1.60 (m, 1H), 4.62 (s, 2H), 5.02 (m, 1H), 6.81 (m, 1H), 6.92 (m, 1H), 7.16 (m, 1H), 7.24 (m, 1), 7.31 (m, 1H), 7.80 (m, 1H), 7.82 (m, 1H), 7.84 (m, 1H), 8.05 (m, 2H), 8.04 (m, 1H), 8.14-8.36 (m, 2H). MS APCI, m/z=454 (M+1). LCMS: 2.42 min.

For example 5 and 6, the starting acid, 3-(cyanomethy)-2-(3-fluorophenyl)quinoline-4-carboxylic acid (1c′), was prepared using a procedure similar to that described in Example 1, using methyl 3-(bromomethyl)-2-(3-fluorophenyl)quinoline-4-carboxylate as the starting material, the compound (1c′) was obtained as a white solid (232 mg, 80%). ¹H NMR (300 MHz, CDCl₃) δ 4.65 (s, 2H), 6.81 (m, 1H), 7.15 (m, 1H), 7.79 (m, 2H), 7.91 (m, 1H), 8.11 (m, 1H), 8.35 (m, 1H), 8.37 (m, 1H), 8.59 (m, 1H). MS APCI, m/z=307 (M+1). LCMS: 1.22 min.

Other compounds include:
methyl 2-(3-(cyanomethyl)-2-phenylquinoline-4-carboxamido)-2-phenylacetate;
3-(cyanomethyl)-2-phenyl-N-(1-phenylethyl)quinoline-4-carboxamide, and
3-(cyanomethyl)-2-phenyl-N-(1-phenylpropyl)quinoline-4-carboxamide.

### Biological Tests

### NK-3 Receptor Binding Activity:

Generally, NK-3r binding activity may be assessed using assays performed as described in Krause et al., (Proc. Natl. Acad. Sci. USA 94: 310-315, 1997). NK-3r complementary DNA is cloned from human hypothalamic RNA using standard procedures. The receptor cDNA is inserted into a suitable expression vector transfected into a Chinese hamster ovary cell line, and a stably-expressing clonal cell line may be isolated, characterized and used for experiments.

Cells may be grown in tissue culture medium by techniques known to those of skill in the art and recovered by low speed centrifugation. Cell pellets may be homogenized, total cellular membranes isolated by high speed centrifugation and suspended in buffered saline. Generally, receptor binding assays may be performed by incubating suitable amounts of purified membrane preparations with ¹²⁵I-methylPhe7-neurokinin B, in the presence or absence of test compounds. Membrane proteins may be harvested by rapid filtration and radioactivity may be quantitated in a β-plate scintillation counter. Nonspecific binding may be distinguished from specific binding by use of suitable controls and the affinity of compounds for the expressed receptor may be determined by using different concentrations of compounds.

Preparation of Membranes from CHO Cells Transfected with Cloned NK-3 Receptors:

A human NK-3 receptor gene was cloned using methods similar to those described for other human NK receptors (Aharony et al., Mol. Pharmacol. 45:9-19, 1994; Caccese et al., Neuropeptides 33, 239-243, 1999). The DNA sequence of the cloned NK-3 receptor differed from the published sequence (Buell et al., FEBS Letts. 299,90-95, 1992; Huang et al., Biochem. Biophys. Res. Commun. 184,966-972, 1992) having a silent single T>C base change at nucleotide 1320 of the coding sequence. Since the change is silent, the cloned gene provides a primary amino acid sequence for the encoded NK-3 receptor protein identical to the published sequence. The receptor cDNA was used to transfect CHO-K1 cells using standard methods and a clone stably-expressing the receptor was isolated and characterized. Plasma membranes from these cells were prepared as published (Aharony et al., 1994).

Cells were harvested and centrifuged to remove medium. The pelleted cells were homogenized (Brinkman Polytron, three 15 sec bursts on ice) in a buffer consisting of 50 mM Tris-HCl (pH 7.4), 120 mM NaCl, 5 mM KCl, 10 mM EDTA and protease inhibitors (0.1 mg/ml soybean trypsin inhibitor, and 1 mM iodoacetamide). The homogenate was centrifuged at 1000×g for 10 min at 4° C. to remove cell debris. Pellets were washed once with homogenizing buffer. Supernatants were combined and centrifuged at 40,000×g for 20 min at 4° C. The membrane-containing pellet was homogenized with a Polytron as before. The suspension was centrifuged at 40,000×g for 20 min at 4° C., the pellet suspended in buffer (20 mM HEPES, pH 7.4 containing 3 mM MgCl₂, 30 mM KCl, and 100 μM thiorphan) and the protein concentration determined. The membrane suspension was then diluted to 3 mg/ml with buffer containing 0.02% BSA, and flash frozen. Samples were stored at −80° C. until used.

### Assay for NK-3 Receptor Binding Activity:

A receptor binding assay method with [¹²⁵I]-MePhe7-NKB was modified from that described by Aharony et al., J. Pharmacol. Exper. Ther., 274:1216-1221, 1995.

Competition experiments were carried out in 0.2 mL assay buffer (50 mM Tris-HCl, 4 mM MnCl₂, 10 μM thiorphan, pH 7.4) containing membranes (2 μg protein/reaction), tested competitors, and [¹²⁵I]-MePhe7NKB (0.2 nM). Unlabeled homologue ligand (0.5 μM) was used to define nonspecific binding. Incubations were carried out at 25° C. for 90 min. Receptor-bound ligand was isolated by vacuum filtration in a Packard Harvester onto GF/C plates presoaked in 0.5% BSA. Plates were washed with 0.02 M Tris, pH 7.4. Computation of equilibrium binding constants (K_{D} and Ki), receptor density (Bmax), and statistical analysis was carried out as published previously (Aharony et al., 1995) using GraphPad Prism or IDBS XLfit software.

### NK-3 Functional Activity:

Generally, NK-3 functional activity may be assessed by using calcium mobilization assays in stable NK-3r-expressing cell lines. Calcium mobilization induced by the methylPhe7-neurokinin B agonist may be monitored using a FLIPR (Molecular Devices) instrument in the manner described by the manufacturer. Agonists may be added to the cells and fluorescence responses continuously recorded for up to 5 min. The actions of antagonists may be assessed by preincubating cells prior to administration of the methylPhe7-neurokinin B agonist. The action of agonists may be assessed by observing their intrinsic activity in such a system.

### Assay for NK-3 Functional Activity:

NK-3 receptor expressing CHO cells were maintained in growth media (Ham's F12 medium, 10% FBS, 2 mM L-glutamine, and 50 mg/mL Hygromycin B). One day prior to the assay cells were dispensed into 384-well plates in Ultraculture media (Cambrex Bio Science) with 2 mM L-glutamine to achieve 70-90% confluency. To quantify NK-3 receptor-induced calcium mobilization, cells were first washed with assay buffer consisting of Hanks' Balanced Salt Solution, 15 mM HEPES, and 2.5 mM probenecid, pH 7.4. The cells were then loaded with Fluo4/AM dye (4.4 μM) in assay buffer. Cells were incubated for one hour and then washed with assay buffer, exposed to 0.02-300 nM senktide and the fluorescence response recorded using a FLIPR instrument (Molecular Devices Corporation). To quantify antagonism of the agonist response, cells were preincubated with varying concentrations of test compound for 2-20 min and then exposed to 2 nM senktide, a concentration that alone elicits about an 70% maximal calcium response. The resulting data was analyzed using XLfit software (IDBS manufacturer) to determine EC50 and IC50 values.

1. A compound in accord with Formula I 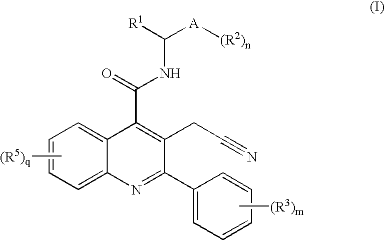
wherein:
R¹ is selected from H, C₁₋₆alkyl-, C₃₋₆cycloalkyl-, C₁₋₆alkyl-C(O)— and C₁₋₄alkylOC(O)—;
A is phenyl or C₃₋₇cycloalkyl-;
n is 1, 2 or 3;
R² at each occurrence is independently selected from H, —OH, —NH₂, —CN, halogen, C₁₋₆alkyl-, C₃₋₇cycloalkyl-, C₁₋₆alkoxy- and C₁₋₆alkoxyC₁₋₆alkyl-;
R³ at each occurrence is independently selected from H, —OH, —NH₂, —NO₂, —CN, halogen, C₁₋₆alkyl-, C₁₋₆alkoxy- and C₁₋₆alkoxyC₁₋₆alkyl-;
m is 1, 2 or 3;
R⁵ at each occurrence is independently selected from H, —OH, —CN, halogen, —R⁶, —OR⁶, —NR⁶R⁷, —SR⁶, —SOR⁶ and —SO₂R⁶;
q is 1, 2or 3;
wherein:
R⁶ and R⁷ at each occurrence are independently selected from H, a C₁₋₆ straight or branched alkyl group, a C₂₋₆ straight or branched alkenyl or alkynyl group and a C₃₋₇carbocyclic group having zero, one or two double- or triple-bonds, wherein said groups are either unsubstituted or substituted with one or more moieties selected from —OH, ═O, —NH₂, —CN, halogen, aryl and C₁₋₃alkoxy-;
and,
when R¹, R² or R³ is an alkyl, cycloalkyl, alkoxy or alkoxyalkyl moiety, said moieties are unsubstituted or have 1, 2, 3, 4 or 5 substituents independently selected at each occurrence from —OH, —NH₂, —CN, phenyl and halogen;
or a stereoisomer, enantiomer, in vivo-hydrolysable precursor or pharmaceutically-acceptable salt thereof.

2. A compound according to claim 1, wherein:
A is phenyl;
R¹ is selected from C₁₋₆alkyl-, C₃₋₆cycloalkyl-, or C₁₋₆alkyl-O—C(O)—;
n at each occurrence is independently selected from 1 or 2;
or a stereoisomer, enantiomer, in vivo-hydrolysable precursor or pharmaceutically-acceptable salt thereof.

3. A compound according to claim 1, wherein:
A is phenyl;
R¹ is selected from C₁₋₆alkyl or —(CO)—O—C₁₋₆alkyl;
n at each occurrence is 1;
or a stereoisomer, enantiomer, in vivo-hydrolysable precursor or pharmaceutically-acceptable salt thereof.

4. A compound according to claim 1, selected from:
3-(Cyanomethyl)-2-phenyl-N-[(1S)-1-phenylpropyl]quinoline-4-carboxamide;
3-(Cyanomethyl)-2-phenyl-N-[(1S)-1-phenylethyl]quinoline-4-carboxamide;
Methyl(2R)-({[3-(cyanomethyl)-2-phenylquinoline-4-yl]carbonyl}amino)(phenyl)acetate;
3-(Cyanomethyl)-N-[(S)-cyclopropyl(3-fluorophenyl)methyl]-2-phenylquinoline-4-carboxamide;
3-(Cyanomethyl)-2-(3-fluorophenyl)-N-[(1S)-1-phenylpropyl]quinoline-4-carboxamide;
3-(Cyanomethyl)-N-[(S)-cyclopropyl(3-fluorophenyl)methyl]-2-(3-fluorophenyl)quinoline-4-carboxamide;
Methyl2-(3-(cyanomethyl)-2-phenylquinoline-4-carboxamido)-2-phenylacetate;
3-(Cyanomethyl)-2-phenyl-N-(1-phenylethyl)quinoline-4-carboxamide, and
3-(Cyanomethyl)-2-phenyl-N-(1-phenylpropyl)quinoline-4-carboxamide;
or a stereoisomer, enantiomer, in vivo-hydrolysable precursor or pharmaceutically-acceptable salt thereof.

5. A compound according to claim 1, in accord with Formula III 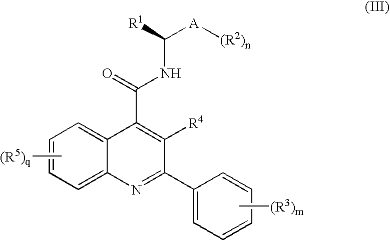
wherein R¹, A, R², n, R³, m, R⁴, R⁵ and q are as defined for Formula I;
or a stereoisomer, in vivo-hydrolysable precursor or pharmaceutically-acceptable salt thereof.

6-16. (canceled)

17. A compound according to claim 5,
wherein:
A is phenyl;
R¹ is selected from C₁₋₆alkyl or —(CO)—O—C₁₋₆alkyl, and
R², R³ and R⁵ are each H;
or a stereoisomer, enantiomer, in vivo-hydrolysable precursor or pharmaceutically-acceptable salt thereof.

18. A compound according to claim 17 that is methyl-(2R)-({[3-(cyanomethyl)-2-phenylquinoline-4-yl]carbonyl}amino)(phenyl)acetate or a in vivo-hydrolysable precursor or pharmaceutically-acceptable salt thereof.

19. A pharmaceutical composition comprising a pharmaceutically-acceptable diluent, lubricant or carrier and a compound according to claim 1, or a stereoisomer, enantiomer, in vivo-hydrolysable precursor or pharmaceutically-acceptable salt thereof.

20. A process for preparing a compound in accord with formula I, 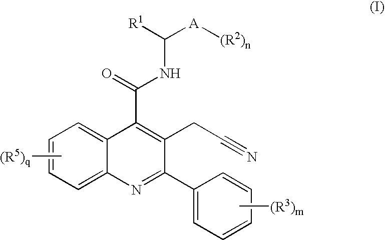
wherein:
R¹ is selected from H, C₁₋₆alkyl-, C₃₋₆cycloalkyl-, C₁₋₆alkyl-C(O)— and C₁₋₄alkylOC(O)—;
A is phenyl or C₃₋₇cycloalkyl-;
n is 1, 2 or 3;
R at each occurrence is independently selected from H, —OH, —NH₂, —CN, halogen, C₁₋₆alkyl-, C₃₋₇cycloalkyl-, C₁₋₆alkoxy- and C₁₋₆alkoxyC₁₋₆alkyl-;
R³ at each occurrence is independently selected from H, —OH, —NH₂, —NO₂, —CN, halogen, C₁₋₆alkyl-, C₁₋₆alkoxy- and C₁₋₆alkoxyC₁₋₆alkyl-;
m is 1, 2 or 3;
R⁵ at each occurrence is independently selected from H, —OH, —CN, halogen, —R⁶, OR⁶, —NR⁶R⁷, —SR⁶, —SOR⁶ and —SO₂R⁶;
q is 1, 2 or 3;
wherein:
R⁶ and R⁷ at each occurrence are independently selected from H, a C₁₋₆ straight or branched alkyl group, a C₂₋₆ straight or branched alkenyl or alkynyl group and a C₃₋₇carbocyclic group having zero, one or two double- or triple-bonds, wherein said groups are either unsubstituted or substituted with one or more moieties selected from —OH, ═O, —NH₂, —CN, halogen, aryl and C₁₋₃alkoxy-;
and,
when R¹, R² or R³ is an alkyl, cycloalkyl, alkoxy or alkoxyalkyl moiety, said moieties are unsubstituted or have 1, 2, 3, 4 or 5 substituents independently selected at each occurrence from —OH, —NH₂, —CN, phenyl and halogen;
said process comprising:
reacting a bromomethyl substituted quinoline ester 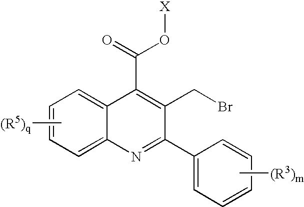
where X is an alkyl moiety, with sodium cyanide in DMF to form a nitrile 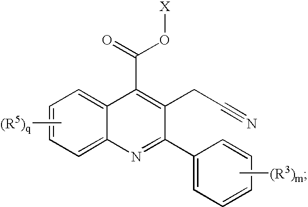
reacting said nitrile with LiOH in THF/water solvent to form an acid 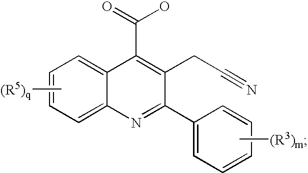
coupling said acid to an amine 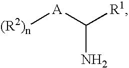
by reacting with (N-(3-dimethylaminopropyl)-N′-ethylcarbodiimide), hydroxybenzotriazole and morpholine in a CH₂Cl₂ solution to form said compound of Formula I.

21. A method of treatment or prophylaxis of a disease or condition in which modulation of the NK3 receptor is beneficial selected from:
depression, anxiety, schizophrenia, cognitive disorders, psychoses, obesity, inflammatory diseases, irritable bowel syndrome, inflammatory bowel disorder, emesis, pre-eclampsia, chronic obstructive pulmonary disease, disorders associated with excessive gonadotrophins, disorders associated with excessive androgens, dysmenorrhea, benign prostatic hyperplasia, prostatic cancer, and testicular cancer;
which method comprises:
administering to a subject suffering from said disease or condition a therapeutically-effective amount of a compound in accord with formula I, 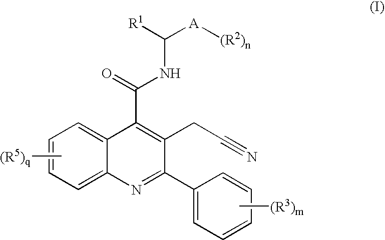
wherein:
R¹ is selected from H, C₁₋₆alkyl-, C₃₋₆cycloalkyl-, C₁₋₆alkyl-C(O)— and C₁₋₄alkylOC(O)—;
A is phenyl or C₃₋₇cycloalkyl-;
n is 1, 2 or 3;
R² at each occurrence is independently selected from H, —OH, —NH₂, —CN, halogen, C₁₋₆alkyl-, C₃₋₇cycloalkyl-, C₁₋₆alkoxy- and C₁₋₆alkoxyC₁₋₆alkyl-;
R³ at each occurrence is independently selected from H, —OH, —NH₂, —NO₂, —CN, halogen, C₁₋₆alkyl-, C₁₋₆alkoxy- and C₁₋₆alkoxyC₁₋₆alkyl-;
m is 1, 2 or 3;
R⁵ at each occurrence is independently selected from H, —OH, —CN, halogen, —R⁶, —OR⁶, —NR⁶R⁷, —SR⁶, —SOR⁶ and —SO₂R⁶;
q is 1, 2or3;
wherein:
R⁶ and R⁷ at each occurrence are independently selected from H, a C₁₋₆ straight or branched alkyl group, a C₂₋₆ straight or branched alkenyl or alkynyl group and a C₃₋₇carbocyclic group having zero, one or two double- or triple-bonds, wherein said groups are either unsubstituted or substituted with one or more moieties selected from —OH, ═O, —NH₂, —CN, halogen, aryl and C₁₋₃alkoxy-;
and,
when R¹, R² or R³ is an alkyl, cycloalkyl, alkoxy or alkoxyalkyl moiety, said moieties are unsubstituted or have 1, 2, 3, 4 or 5 substituents independently selected at each occurrence from —OH, —NH₂, —CN, phenyl and halogen;
or a stereoisomer, enantiomer, in vivo-hydrolysable precursor or pharmaceutically-acceptable salt thereof.

22. A method according to claim 21, wherein said disease or condition is selected from anxiety, depression, schizophrenia or obesity.

23. A method according to claim 21, in which said compound is a compound accord with Formula III 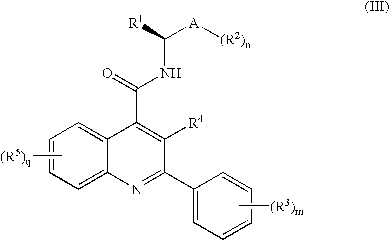
wherein R¹, A, R², n, R³, m, R⁴, R⁵ and q are as defined for Formula I;
or a stereoisomer, in vivo-hydrolysable precursor or pharmaceutically-acceptable salt thereof.

24. A method according to claim 23,
wherein in said compound in accord with Formula III:
A is phenyl;
R¹ is selected from C₁₋₆alkyl or —(CO)—O—C₁₋₆alkyl, and
R², R³ and R⁵ are each H;
or a stereoisomer, enantiomer, in vivo-hydrolysable precursor or pharmaceutically-acceptable salt thereof.

25. A method according to claim 24, wherein said compound in accord with Formula III is methyl-2R)-({[3-(cyanomethyl)-2-phenylquinoline-4-yl]-carbonyl}amino)(phenyl)acetate or a in vivo-hydrolysable precursor or pharmaceutically-acceptable salt thereof.

26. A method of treatment or prophylaxis of a disease or condition in which modulation of the NK3 receptor is beneficial selected from:
depression, anxiety, schizophrenia, cognitive disorders, psychoses, obesity, inflammatory diseases, irritable bowel syndrome, inflammatory bowel disorder, emesis, pre-eclampsia, chronic obstructive pulmonary disease, disorders associated with excessive gonadotrophins, disorders associated with excessive androgens, dysmenorrhea, benign prostatic hyperplasia, prostatic cancer, and testicular cancer;
which method comprises:
administering to a subject suffering from said disease or condition a therapeutically effective amount of a pharmaceutical composition comprising a compound in accord with formula I, 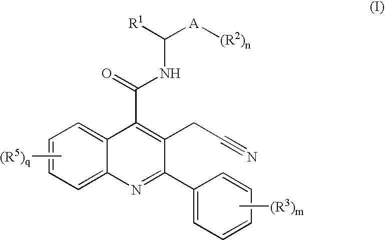
wherein:
R¹ is selected from H, C₁₋₆alkyl-, C₃₋₆cycloalkyl-, C₁₋₆alkyl-C(O)— and C1-4alkylOC(O)—;
A is phenyl or C₃₋₇cycloalkyl-;
n is 1, 2 or3;
R² at each occurrence is independently selected from H, —OH, —NH₂, —CN, halogen, C₁₋₆alkyl-, C₃₋₇cycloalkyl-, C₁₋₆alkoxy- and C₁₋₆alkoxyC₁₋₆alkyl-;
R³ at each occurrence is independently selected from H, —OH, —NH₂, —NO₂, —CN, halogen, C₁₋₆alkyl-, C₁₋₆alkoxy- and C₁₋₆alkoxyC₁₋₆alkyl-;
m is 1, 2 or 3;
R⁵ at each occurrence is independently selected from H, —OH, —CN, halogen, —R⁶, —OR⁶, —NR⁶R⁷, —SR⁶, —SOR⁶ and —SO₂R⁶;
q is 1, 2 or 3;
wherein:
R⁶ and R⁷ at each occurrence are independently selected from H, a C₁₋₆ straight or branched alkyl group, a C₂₋₆ straight or branched alkenyl or alkynyl group and a C₃₋₇carbocyclic group having zero, one or two double- or triple-bonds, wherein said groups are either unsubstituted or substituted with one or more moieties selected from —OH, ═O, —NH₂, —CN, halogen, aryl and C₁₋₃alkoxy-;
and,
when R¹, R² or R³ is an alkyl, cycloalkyl, alkoxy or alkoxyalkyl moiety, said moieties are unsubstituted or have 1, 2, 3, 4 or 5 substituents independently selected at each occurrence from —OH, —NH₂, —CN, phenyl and halogen
and a pharmaceutically-acceptable diluent, lubricant or carrier.

27. A method according to claim 26, wherein said disease or condition is selected from anxiety, depression, schizophrenia or obesity.

28. A method according to claim 26, in which said compound is a compound accord with Formula III 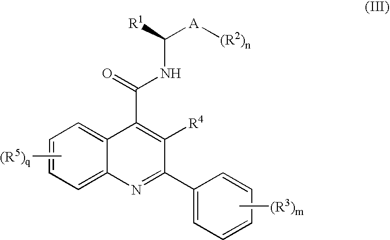
wherein R¹, A, R², n, R³, m, R⁴, R⁵ and q are as defined for Formula I;
or a stereoisomer, in vivo-hydrolysable precursor or pharmaceutically-acceptable salt thereof.

29. A method according to claim 28,
wherein in said compound in accord with Formula III:
A is phenyl;
R¹ is selected from C₁₋₆alkyl or —(CO)—O—C₁₋₆alkyl, and
R², R³ and R⁵ are each H;
or a stercoisomer, enantiomer, in vivo-hydrolysable precursor or pharmaceutically-acceptable salt thereof.

30. A method according to claim 29, wherein said compound in accord with Formula III is methyl-2R)-({[3-(cyanomethyl)-2-phenylquinoline-4-yl]-carbonyl}amino)(phenyl)acetate or a in vivo-hydrolysable precursor or pharmaceutically-acceptable salt thereof.
